(19) United States
(12) Patent Application Publication
(10) Pub. No.: US 2022/0233786 A1
(43) Pub. Date: Jul. 28, 2022

(54) NEEDLE COVER AND SAFETY NEEDLE ASSEMBLY INCLUDING SAME

(71) Applicant: METIER MEDICAL LIMITED, Cessnock,New South Wales (AU)

(72) Inventors: Krzysztof NIESIOBEDZKI, Hamburg (DE); Larry MINIX, Pointe Vedra Beach, FL (US); Grant Raymond HOWELLS, New South Wales (AU)

(21) Appl. No.: 17/614,937

(22) PCT Filed: May 29, 2020
(86) PCT No.: PCT/AU2020/050545

§ 371 Date: Nov. 29, 2021

(30) Foreign Application Priority Data
May 31, 2019 (AU) 2019901877

(51) Int. Cl. A61M 5/32

(52) U.S. Cl. CPC A61M 5/3216; A61M 2205/273; A61M 2005/3217

(57) ABSTRACT

A needle cover including: a mounting portion for mounting a needle; and a lid portion hingedly connected to the mounting portion, the lid portion movable to: an open position; a first cover position, wherein the lid portion is releasably docked with the mounting portion; and a second cover position, wherein the lid portion is irreversibly docked with the mounting portion, wherein the mounting portion and the lid portion include corresponding docking elements that engage to provide the first and second cover positions.

### FIELD OF THE INVENTION

The present invention relates to needle covers, and, in particular, to needle covers that are implemented to minimize needle stick injury for healthcare workers.

### BACKGROUND OF THE INVENTION

Syringes are common place in the medical industry. They are routinely used by doctors, nurses and other medical professionals for administering drugs and collecting blood. There is always a risk of needle stick injury for healthcare workers when using a syringe. Apart from physical injury, needle stick injury puts workers at risk of contracting disease from patients.

One approach to minimizing needle stick injury is to implement a hinged needle cap, which opens to expose the needle for use, and thereafter, can be permanently closed such that needle tip is enclosed/concealed, and can be disposed of in a safe manner. However, current hinged needle caps have several disadvantages.

In the first instance, existing caps generally only have an open and a permanently closed position, there is no intermediate cover position where the needle tip may be covered temporarily. In hospitals today, it is not uncommon to draw medication into a syringe at a location away from the patient, however, after this, the healthcare worker must travel to the patient, who may be located some distance away, with the needle tip exposed. As closure of the cap is configured to be permanent, it is not an option to close the cap, and the worker is at risk.

Existing hinged caps are also limited in that, if holding a syringe in one hand, a healthcare worker is required to use their opposite hands to close the cap. This closure action, which often needs to be carried out with some force, also puts the healthcare worker at risk, particularly when they are tired and not concentrating.

The present invention seeks to address disadvantages associated with conventional needle caps.

Any reference herein to known prior art does not, unless the contrary indication appears, constitute an admission that such prior art is commonly known by those skilled in the art to which the invention relates, at the priority date of this application.

### SUMMARY OF THE INVENTION

In one broad form, the present invention provides, a needle cover including: a mounting portion for mounting a needle; and a lid portion hingedly connected to the mounting portion, the lid portion movable to: an open position; a first cover position, wherein the lid portion is releasably docked with the mounting portion; and a second cover position, wherein the lid portion is irreversibly docked with the mounting portion, wherein the mounting portion and the lid portion include corresponding docking elements that engage to provide the first and second cover positions.

In one form, the corresponding docking elements include at least one docking projection and docking port pair, the docking projection configured for engagement with the docking port.

In one form, the docking projection includes a distal hook portion and a proximal hook portion, the distal hook portion for engagement with the docking port to provide the first cover position, and the proximal hook portion for engagement with the docking port to provide the second cover position. In one form, the hook portions are provided by lateral expansions on the docking projection. In one form, the largest lateral radius of the proximal hook portion is larger than the largest lateral radius of the distal hook portion.

In one form, the docking port includes one or more resiliently deformable elements that deform to allow entry of the hook portions, and engage with the hook portions to provide the first and second cover positions. In one form, the docking port includes an aperture with resiliently deformable elements in the form of internally projecting tabs.

In one form, in the first cover position, the docking projection is engaged with the docking port such that the distal hook portion thereof is inserted beyond the tabs and the proximal hook portion is not, and, the tabs are configured to resist withdrawal of the distal hook portion.

In one form, in the second cover position, the docking projection is engaged with the docking port such that the distal hook portion and the proximal hook portion are both inserted beyond the tabs, and, the tabs are configured to prohibit withdrawal of the proximal hook portion.

In one form, corresponding docking elements are respectively located on substantially flat closure flanges of the mounting portion and the lid portion. In one form, in the second cover position, the closure flanges of the mounting portion and the lid portion come into face-to-face contact. In one form, in the first cover position, the closure flanges are at an angle with respect to one another. In one form, the closure flanges include a hinge therebetween to provide the hinged connection between the mounting portion and the lid portion of the needle cover. In one form, the corresponding docking elements are located adjacent a side edge of the closure flanges that is distal or opposite to the hinge.

In one form, the hinged connection is configured to resist movement from a fully open positional range, wherein the opening angle between the mounting and lid portions is greater than about 180 degrees, to a partly open positional range, wherein the opening angle mounting and lid portions is less than about 180 degrees. In one form, in the partly open positional range, the hinge connection is configured such that the lid portion is biased towards the first cover position.

In one form, the lid portion includes an elongate groove for covering any mounted needle. In one form, the elongate groove includes at least one retaining element therein to irreversibly secure the needle within the groove. In one form, the elongate groove includes three retaining elements spaced along its length.

In one form, the lid portion includes a beveled tip.

In a further broad form, the present invention provides a safety needle assembly for mounting to a syringe barrel, the safety needle assembly including: a needle cover as described in any one of the above forms; and a needle mounted to the mounting portion.

### BRIEF DESCRIPTION OF THE DRAWINGS

A detailed description of a preferred embodiment will follow, by way of example only, with reference to the accompanying drawings, in which:

FIG. 1 is a side view of a needle cover/safety needle according to one example of the invention, with lid portion in the open position;

FIG. 2 is a side view of the needle cover/safety needle of FIG. 1, with lid portion in a first cover position;

FIG. 3 is a cross sectional side view of FIG. 2, showing placement of the needle shaft when lid portion is in the first cover position;

FIG. 4 is a cross sectional side view of the needle cover/safety needle of FIG. 1 when the lid portion is in a second cover position, showing placement of the needle shaft;

FIG. 5 is front perspective of the needle cover/safety needle of FIG. 1, with the lid portion in an open position;

FIG. 6 is a top perspective view of the needle cover/safety needle of FIG. 1, with the lid portion in an open position;

FIG. 7 is a side view of the needle cover/safety needle of FIG. 1, with the lid portion in the open position, showing a magnified view of the docking projection;

FIG. 8 is a perspective view of the needle cover/safety needle of FIG. 1, with the lid portion in the open position;

FIG. 9 is a rear view of the needle cover/safety needle of FIG. 1;

FIG. 10 is a perspective view of a needle cover/safety needle according to one example, including a removable sheath on the needle shaft; and

FIG. 11 is a side view of the needle cover/safety needle of FIG. 9, with removable sheath in place.

### DETAILED DESCRIPTION

Embodiments of the invention provide needle covers to protect healthcare workers from needle stick injury. The needle covers as described herein include a mounting portion for mounting a needle, and a lid portion hingedly connected to the mounting portion. It will be appreciated that the mounting portion may be configured to mount different types of needles, needle assemblies, or needle containing devices. For example, the mounting portion may be configured to mount a needle shaft directly or to mount the hub of a needle assembly.

The lid portion is moveable to an open position, where the needle is available/exposed for use, and first and second cover positions, where the needle tip is sufficiently covered/concealed. In the first cover position, the lid portion is releasably docked with the mounting portion such that, when sufficient force is applied, it can return to the open position. In contrast, in the second cover position, the lid portion is irreversibly docked with the mounting portion so that the lid portion cannot return to the open position.

In an example of typical use, a healthcare worker may initially have the lid portion in the open position to draw medication into a syringe connected to the needle cover. Once the syringe is loaded with medication, the lid portion may be moved into the first cover position, whilst the healthcare worker waits for the patient to become available, or, if the healthcare worker needs to walk to the patient. The releasable nature of the first cover position allows that the healthcare worker, once at the patient, is able to uncover the needle to inject the medication into the patient. Once the medication has been administered, the healthcare worker can then move the lid portion into the second cover position, which permanently encloses/closes off/conceals the needle tip, to ensure there is no opportunity for subsequent needle stick injury, and to assist with safe disposal of the needle. Permanent closure of the needle cover also prohibits re-use of the needle, a problem that is particularly widespread in developing nations.

To provide first and second cover positions, the mounting portion and the lid portion include corresponding docking elements that engage with one another. It will be appreciated that the corresponding docking elements may take a variety forms. The corresponding docking elements may, for example, include at least one docking projection and docking port pair, where the docking projection is configured for engagement with the docking port. Generally, the needle cover includes multiple docking projection and docking port pairs.

The docking projection and docking port pair/s may be configured such that there is staged or progressive engagement therebetween, so as to provide the first and second cover positions. For example, the docking projection may include a distal hook portion and a proximal hook portion, the distal hook portion for engagement with the docking port to provide the first cover position and the proximal hook portion for engagement with the docking port to provide the second cover position. For example, with initial insertion of the docking projection into the docking port, the distal hook portion may be engaged to maintain the first cover position, and subsequently, with continued insertion of the docking protection into the port, the proximal portion may be engaged to maintain the second cover position.

It will be appreciated that the hook portions may take a variety of forms and at a minimum may be any form of protruding element/flange configured to hook on to a corresponding portion of the docking port. In one example, the hook portions are provided by lateral expansions on the docking projection. Typically, the lateral expansions expand/extend radially from the docking projection. It will be appreciated that the shape of the expansions may vary, and may, for example, be rounded, conical, frusto-conical, bulbous, pyramid-like, mushroom shaped etc. Generally, the proximal hook portion is larger or more expansive than the distal hook portion. Typically, the largest lateral radius or extension of the proximal hook portion is larger than the largest lateral radius or extension of the distal hook portion. This allows for a different level of engagement with the docking port, which typically includes one or more resiliently deformable elements that deform to allow entry of the hook portions.

In one example form, the docking port includes an aperture with internally projecting tabs that deform to allow entry of the hook portions. The tabs are resiliently deformable such that after the hook portions are advanced beyond the tabs, the tabs return to the non-deformed state to resist or prohibit withdrawal of the hook portions.

For the first cover position, the docking projection is engaged with the docking port such that only the distal hook portion thereof is inserted beyond the tabs (i.e. the proximal hook portion is not), and the tabs are configured to resist withdrawal of the distal hook portion. In the second cover position, the docking projection is engaged with the docking port such that the distal hook portion and the proximal hook portion are both inserted beyond the tabs, and the tabs are configured to prohibit withdrawal of the proximal hook portion.

Whilst it will be appreciated that the mounting portion and the lid portion may take a variety of forms, the corresponding docking elements are generally located on substantially flat closure flanges of the mounting portion and the lid portion. The closure flanges being those which come into face-to-face contact when the needle cover is in the permanently closed or second cover position. In the first cover position, where the docking projections are typically not fully inserted into the respective docking ports, the closure flanges are at an angle with respect to one another.

Generally, the hinged connection between the mounting portion and the lid portion of the needle cover is provided by a hinge between the closure flanges of the mounting portion and the lid portion. The corresponding docking elements are generally located adjacent the side edges of the closure flanges that are distal from or opposite to the hinge. Having the corresponding docking elements distal to the hinge provides that the angle of entry of any docking projection is shallower, facilitating entry into the docking port.

The hinged connection between the mounting and lid portions may be, for example, active such that it is biased to certain positions. In one form, the hinged connection may be configured to resist movement from a fully open positional range, wherein the opening angle between mounting portion and lid portion is greater than about 180 degrees, to a partly open positional range, wherein the opening angle between mounting portion and lid portion is less than about 180 degrees.

Generally, once in the partly open positional range (i.e. where the angle between the mounting portion and lid portion is less than about 180 degrees), the hinged connection is configured such that the lid portion is biased towards the first cover position. Conversely, in the fully open positional range, the lid portion is biased to a fully open or maximally extended position (i.e. in use the fully open or maximally extended position would have the lid resting up against the syringe barrel).

As an example of how the hinged connection may operate, a healthcare worker may initially have the lid portion in a fully open or maximally extended position, i.e. where the lid portion rests up against syringe barrel to which the needle cover is mounted. After administering the injection, the healthcare worker, using the same hand that is holding syringe, may begin to move the lid portion towards the first cover position. As the lid portion passes the changeover point (e.g. typically where the opening angle between the mounting and lid portion is about 180°), the hinged connection is configured such that the lid portion is automatically advanced towards the first cover position, thus allowing the healthcare worker to remove their finger from the lid portion, and minimize the risk of needle stick injury. The remainder of movement of the lid portion into the first cover position, can be then carried out by applying force to the lid portion using a nearby surface. To facilitate this, the lid portion may include a beveled tip.

The lid portion generally includes an elongate groove that houses or covers the needle in the first and second cover positions. To reinforce the second cover position, the elongate groove may include at least one retaining element therein to irreversibly secure the needle within the groove. Typically, the elongate groove includes three retaining elements spaced along its length.

As described, the needle cover, via the mounting portion, may be configured fit any type of needle/needle assembly/needle containing device. It will also be appreciated that the needle covers as described herein may form part of a safety needle assembly that includes a needle shaft mounted within the needle cover. The safety needle assembly being such that it can be connected directly to a syringe barrel for use (e.g. on to a conventional syringe barrel needle adaptor). It will also be appreciated that the needles covers as described herein may be (and are typically) formed of a suitable polymer/plastic material.

An additional needle sheath may also be used in conjunction with the needle covers as described herein, which would cover the needle prior to use. For example, in the packaged or ‘as provided’ form, a safety needle assembly including the needle cover as described herein may include an additional needle sheath that covers the needle, and prevents the lid portion of the needle cover moving into the first and/or second cover positions. In typical use, healthcare worker would, in the first instance, move the lid portion into the fully open or maximally extended position (e.g. where it rests against syringe barrel), and then, remove the additional sheath to use the needle. Once the sheath is removed, the needle is exposed for use. As the sheath cannot be replaced without substantial risk of needle stick injury, the lid portion of the needle cover is utilized, by moving it into the first and/or second cover positions as required.

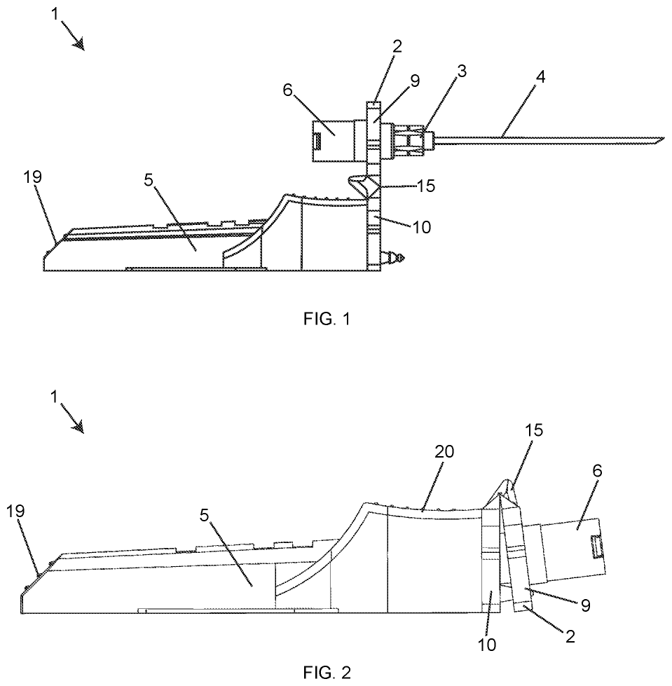

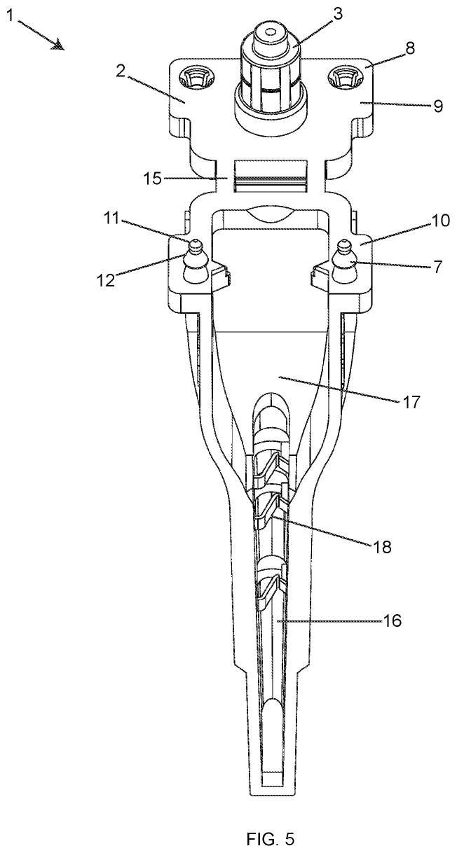

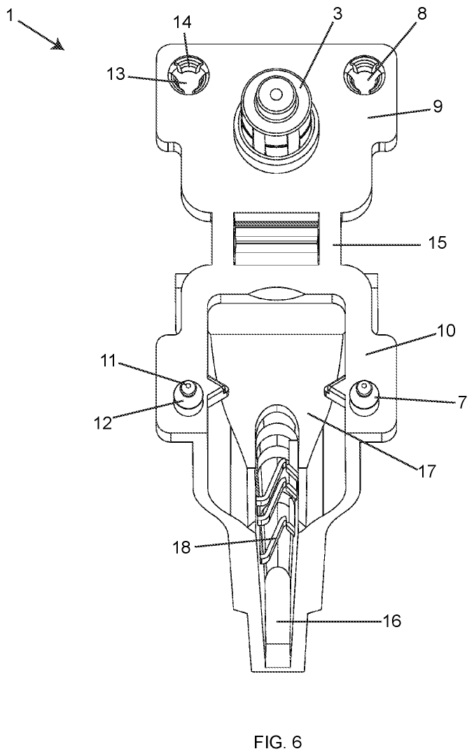

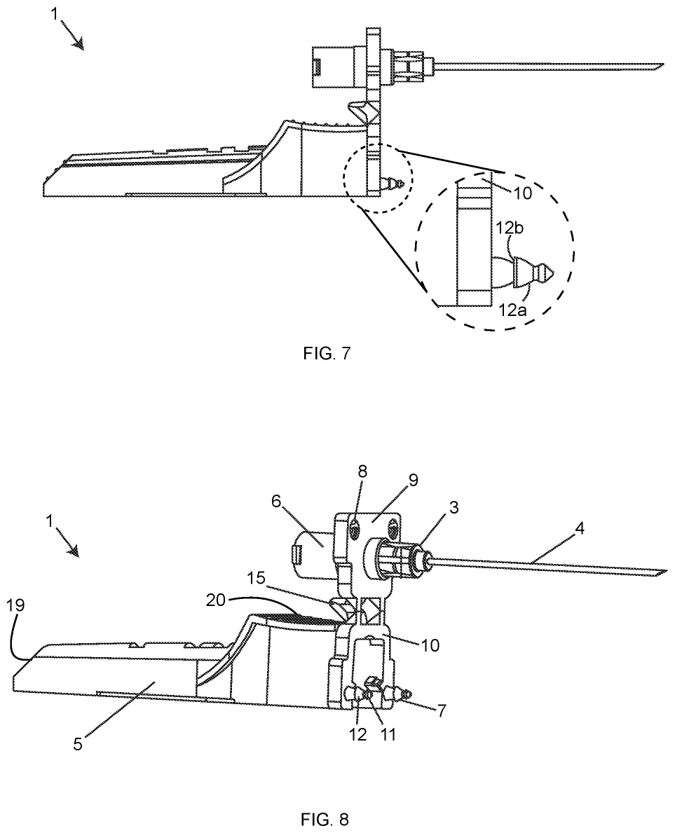

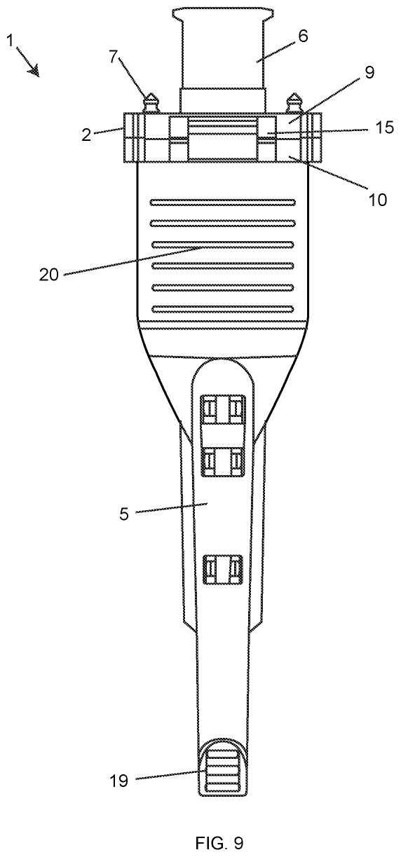

One particular form of a needle cover (1) according to the invention is shown in FIGS. 1 to 6 as part of a safety needle assembly. The needle cover (1) includes a mounting portion (2) with a hub (3), to which a needle shaft (4) is mounted. A lid portion (5) is hingedly connected to the mounting portion (2), and is moveable to cover the needle shaft (4). The mounting portion (2) also includes a syringe connection element (6) that is configured to receive a needle adapter portion of a syringe barrel (not shown).

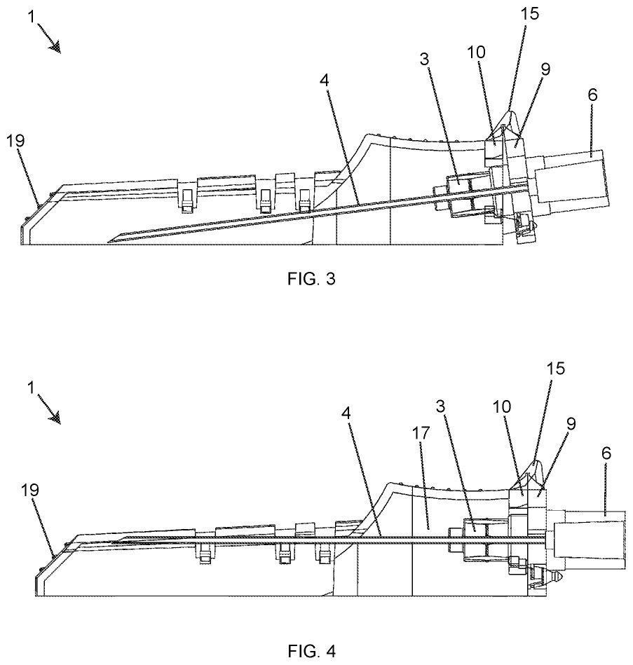

From the open position (see FIG. 1), the lid portion (5) is movable into a first cover position, wherein the lid portion (5) is releasably docked with the mounting portion (2). The first cover position is shown in FIGS. 2 and 3, which show an external side view and a cross-sectional side view respectively. In typical use, the first cover position is used when the needle needs to be covered temporarily, such as, for example, when medication has been drawn into the syringe at location away from a relevant patient. This is not uncommon in some hospitals where medications are held and monitored in a centralised pharmacy/store.

The lid portion (5) of the needle cover (1) is also movable into a second cover position, wherein the mounting portion (2) and the lid portion (5) are irreversibly docked together (see FIG. 4). In typical use, the second cover position, or permanently closed position, is used after administering/injecting medication, where the needle is not to be used again. The second cover position, permanently covers/conceals the needle shaft (4) such that it can be disposed of safely.

The first and second cover positions are provided by engagement between corresponding docking elements (7, 8) located on the mounting portion (2) and the lid portion (5). In particular, the mounting portion (2) includes docking ports (8) that are configured to receive docking projections (7) located on the lid portion (5). It will be appreciated that in alternate forms, the docking projections may be located on the mounting portion and the docking ports located on the lid portion, or there may be a mixture of projections and ports on each of the mounting and lid portions.

The docking elements (7, 8) are respectively located on substantially flat closure flanges (9, 10) of the mounting and lid portions (2, 5). In the first cover position (e.g. FIGS. 2 and 3), the closure flanges (9, 10) are angled with respect to one another, whilst in the second cover position, which is a permanently closed position of the needle cover (1), the closure flanges (9, 10) are in face-to-face contact. The hinge connection between the mounting and lid portions is provided by hinge (15) connecting the closure flanges (9, 10).

The docking projections (7), in this example, extend substantially perpendicular to the closure flange (10) of the lid portion (5). The docking projections (7) include distal and proximal hook portions (11, 12) that are configured to engage with the docking ports (8) to provide the first and second cover positions. The docking ports (8) comprise apertures (13) that extend through the closure flange (9) of the mounting portion (2). Resiliently deformable tabs (14) extend internally from the internal walls (15) of the aperture (13), and deform to allow insertion of the hook portions (11, 12).

For the first cover position, only the distal hook portion (11) is advanced beyond the tabs (14). Thereafter, the tabs (14), which are resilient, return to their non-deformed state, and resist withdrawal of the distal hook portions (11). The distal hook portions (11) are sized/shaped such that, with sufficient force, they can be withdrawn, allowing the lid portion (5) to move back into the open position. In this example, the distal hook portions (11) are substantially teardrop shaped. The first cover position, thus provides a temporary covered state.

The second cover position, on the other hand, provides for permanent docking of the lid portion (5) to the mounting portion (2) such that the needle (4) is permanently cover/concealed/enclosed therein. The second cover position is achieved by further advancing the docking projections (7) into the docking ports (8) from the first cover position. In particular, for the second cover position, the proximal hook portions (12) are advanced beyond the tabs (14). The proximal portions (12) have a lateral expansion that is substantially larger than the distal hook portions (11), which provides that a larger amount of force is required to advance the proximal hook portions (12) beyond the tabs (14). The proximal hook portions (12) have a substantially conical shape. The tapered shape/nature of the proximal hook portions (12), which have a leading surface (12a) that is sloped (i.e. with respect to the direction of insertion) when compared to the trailing surface (12b) (which is substantially perpendicular to the direction of insertion), provides that, whilst the proximal hook portion (12) can be readily advanced beyond the tabs (14) of the docking ports (8), they cannot be withdrawn thereafter. Thus there is permanent docking between the lid portion (5) and the mounting portion (2) in the second cover positon. As shown, the distal hook portions (11) have a smaller lateral expansion and are more rounded, such that their engagement with the tabs (14) is readily releasable when sufficient force is applied.

The tabs (14), to facilitate insertion on the one hand and resist/prohibit withdrawal on the other hand, are angled backward (i.e. into the aperture). This provides a sloped entry surface which guides entry of the hook portions (11, 12) into the aperture, and at the same time, provides that tips of the tabs (14) are angled in a backward direction (i.e. in the direction of insertion of the docking projections) that assists with resistance to withdrawal of the hook portions (11, 12).

The docking elements are located on the closure flanges (9, 10) adjacent the edges of the flanges that are distal/opposite to the hinge (15). This is advantageous in that the angle of entry of the projections (7) into the ports (8) is shallower than would otherwise be the case which allows the projections to be longer, and provide multiple docking engagements (i.e. via multiple hook portions).

The flat nature of the closure flanges (9, 10), which come into face-to-face contact in the permanently closed or second cover position, provides a robust engagement between the mounting portion (2) and the lid portion (5), and avoids over rotation of the lid portion (5) when force is applied to move it into second cover position. Over rotation can lead, for example, to the needle shaft penetrating the lid portion, which may increase the risk for injury for a healthcare worker.

As shown, the lid portion (5) includes the elongate groove (16) to house the needle shaft (4). The elongate groove (16) extends from a wider cavity (17) that is able to house the hub (3) when the lid portion (5) is in the first or second cover positions. The elongate groove (16) includes retaining members in the form of resiliently deformable fingers (18), which are configured to permit the needle shaft (4) to advance into the groove but then prevent the needle shaft exiting groove.

FIGS. 3 and 4 show the positioning of the needle shaft (4) in both the first and second cover positions. As shown in FIG. 3, in the first cover position, whilst the distal hook portions of the projections (7) are engaged in the docking port, the needle shaft (4) is not engaged with the deformable fingers (18). This provides that the needle shaft (4) can be re-exposed i.e. the lid portion removed, as required. FIG. 4 on the other hand, shows the second cover position, in which the needle shaft is engaged beyond the deformable fingers (18).

The hinge (15) is active in that it is biased towards certain positions. When the lid portion (5) is in the fully open positional range, i.e. when the angle between the closure flanges (9, 10) is greater than about 180 degrees, the lid portion is biased toward a fully open or maximally extended position, such that, in use, it would typically rest up against the syringe barrel to which the needle cover (1) is mounted. However, as the lid portion (5) is advanced towards the mounting portion (2) the hinge (15) is configured such that the direction of the bias changes once the angle between the closure flanges (9, 10) proceeds below 180°. Thus, while a healthcare worker is initially required to use their thumb or finger to advance the lid portion (5) towards the mounting portion (2), at a certain point, typically when the angle between the closure flanges (9, 10) is about 180 degrees, the lid portion (5) will progress automatically towards the first cover position.

The rear of the lid portion (5) includes ridges (20) thereon to provide grip for the finger/thumb of the healthcare worker. As shown, the ridges (20) are located on a widened finger/thumb pad at the base of the lid portion (5) away from the needle tip. The widened finger/thumb pad area reduces the risk finger/thumb slippage when a healthcare worker is initially advancing the lid portion (5) (i.e. before any automatic advancement provided by the hinge).

After any automatic advancement provided by the active hinge (1), the projections (7) typically rest on or are very close to the ports (8), but the distal hook portions (11) are not yet engaged/inserted therein. For the lid portion (5) to fully enter the first cover position, such that the distal hook portions (11) are inserted/engaged, the healthcare worker may apply a closing force to the lid portion by pressing the lid portion (5) onto a nearby surface. To ensure that force is transmitted effectively and docking is achieved readily, the tip of the lid portion (15) beveled to provide a surface contacting portion (19). The surface contact portion (19) may include ridges in seeking to minimize sliding thereof along the nearby surface as closing force is applied. By using a nearby surface, the healthcare worker does not need to bring their fingers near to the needle shaft in seeking to move the lid portion (5) in to the first or second cover positions.

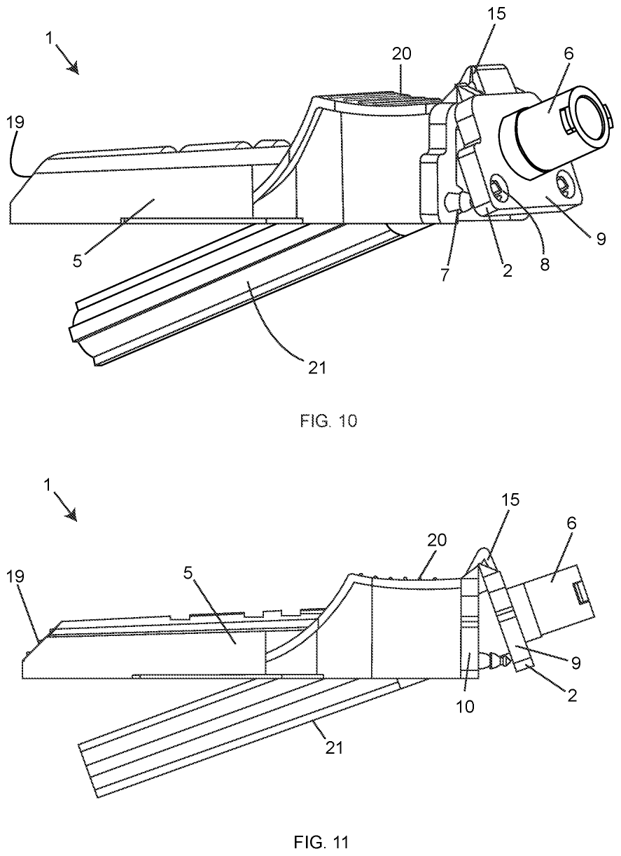

It will be appreciated that the needle cover (1) is typically formed of a suitable polymer/plastic material. FIGS. 10 and 11 illustrate how an additional removable sheath (21) may also be included. As previously mentioned, the as ‘as sold’ or packaged form of the safety needle may include an additional sheath (21) over the needle shaft which is to be removed before use.

It will be appreciated that the presently described needle covers provide several advantages over conventional covers/caps. In particular, by having the lid portion movable into a first, temporary cover position, a healthcare worker is able to temporarily cover the needle shaft/tip when required. In addition, the beveled tip of the lid portion, allows a healthcare worker to more effectively use a nearby surface to close the lid portion, and avoids them having to use their own hand/fingers/thumb.

Where ever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

It will be understood that the invention disclosed and defined herein extends to all alternative combinations of two or more of the individual features mentioned or evident from the text. All of these different combinations constitute various alternative aspects of the invention.

While particular embodiments of this invention have been described, it will be evident to those skilled in the art that the present invention may be embodied in other specific forms without departing from the essential characteristics thereof. The present embodiments and examples are therefore to be considered in all respects as illustrative and not restrictive, and all modifications which would be obvious to those skilled in the art are therefore intended to be embraced therein.

1. A needle cover including:
a mounting portion for mounting a needle having a tip; and
a lid portion hingedly connected to the mounting portion such that the lid and mounting portions can adopt a plurality of positions relative to one another including an open position in which the needle tip, when the needle is mounted to the mounting portion, is exposed for use, and first and second cover positions in which the needle tip, when the needle is mounted to the mounting portion, is covered by the lid portion,
wherein:
in the first cover position the lid and mounting portions are releasably docked together;
in the second cover position the lid and mounting portions are irreversibly docked together so that relative movement therebetween is prevented;
the lid and mounting portions each include a closure flange with a substantially flat face, wherein the faces of the closure flanges are in face-to-face contact in the second cover position and are inclined at an angle with respect to one another in the first cover position;
the lid and mounting portions include cooperating docking elements to dock the lid and mounting portions together in either the first or second cover positions;
the cooperating docking elements include at least one docking projection on the closure flange of one of the lid or mounting portion, the projection extending from the face of the closure flange at a substantially right angle thereto, the projection comprising a distal hook portion and a proximal hook portion;
the cooperating docking elements also include an aperture for each docking projection in the closure flange of the other of the lid or mounting portion, wherein at least one resiliently deformable element extends inwardly from an internal wall of the aperture, the resiliently deformable element being configured to deform to allow entry therebeyond of either the distal or proximal hook portion into the aperture;
the distal hook portion is sized and configured so that it can be inserted into the aperture beyond the at least one resiliently deformable element and subsequently withdrawn therefrom, insertion of only the the distal hook portion into the aperture thereby providing the first cover position; and
the proximal hook portion is sized and configured so that it can be inserted into the aperture beyond the at least one resiliently deformable element but so that it cannot be withdrawn therefrom, insertion of the proximal hook portion into the aperture thereby providing the second cover position.

2. A needle cover as claimed in claim 1, wherein the proximal and distal hook portions each include lateral expansions thereon, and wherein the lateral expansion of the proximal hook portion includes a cross-sectional dimension which is greater than any cross-sectional dimension of the lateral expansion of the distal hook portion.

3. A needle cover as claimed in claim 2, wherein the lateral expansion of the proximal hook portion is of a substantially conical profile and the lateral expansion of the distal hook position has a curved profile.

4. A needle cover as claimed in claim 1, wherein the closure flanges include a hinge therebetween to provide the hinged connection between the mounting portion and the lid portion of the needle cover.

5. A needle cover as claimed in claim 4, wherein the corresponding docking elements are located adjacent a side edge of the closure flanges that is distal or opposite to the hinge.

6. A needle cover as claimed in claim 1, wherein the hinged connection is configured to resist movement from a fully open positional range, wherein the opening angle between the mounting and lid portions is greater than about 180 degrees, to a partly open positional range, wherein the opening angle mounting and lid portions is less than about 180 degrees.

7. A needle cover as claimed in claim 6, wherein, in the partly open positional range, the hinge connection is configured such that the lid portion is biased towards the first cover position.

8. A needle cover as claimed in claim 1, wherein there is provided a plurality of said resiliently deformable elements arranged around the internal wall of the aperture.

9. A needle cover as claimed in claim 1, wherein the lid portion includes an elongate groove for covering any mounted needle.

10. A needle cover as claimed in claim 9, wherein the elongate groove includes at least one retaining element therein to irreversibly secure the needle within the groove.

11. A needle cover as claimed in claim 10, wherein the elongate groove includes three retaining elements spaced along its length.

12. A needle cover as claimed in claim 1, wherein the lid portion includes a beveled tip.

13. A safety needle assembly for mounting to a syringe barrel, the safety needle assembly including:
a needle cover as claimed in claim 1; and
a needle mounted to the mounting portion.
